(19) United States
(12) Patent Application Publication
(10) Pub. No.: US 2013/0075063 A1
(43) Pub. Date: Mar. 28, 2013

(54) AZEOTROPE-LIKE COMPOSITIONS OF CIS-1,3,3,3-TETRAFLUOROPROPENE AND 1,1,1,3,3-PENTAFLUOROPROPANE

(71) Applicants: Ryan Hulse, Getzville, NY (US); Hang T. Pham, Amherst, NY (US); Martin R. Paonessa, N. Niagra Falls, NY (US); Rajiv Ratna Singh, Getzville, NY (US)

(72) Inventors: Ryan Hulse, Getzville, NY (US); Hang T. Pham, Amherst, NY (US); Martin R. Paonessa, N. Niagra Falls, NY (US); Rajiv Ratna Singh, Getzville, NY (US)

(21) Appl. No.: 13/245,477

(22) Filed: Sep. 26, 2011

(51) Int. Cl. F28D 15/00; C09K 3/30; A62C 2/00; C09K 21/08; A61L 2/18; C10M 131/04; C09K 5/04; C08J 9/14

(52) U.S. Cl. 165/104.11; 252/67; 252/182.12; 521/98; 252/601; 422/37; 508/590; 169/46

(57) ABSTRACT

The present invention provides azeotrope-like compositions of cis-1,3,3,3-tetrafluoroprone (HFO-1234ze (Z)) with 1,1,1,3,3-pentafluoropropane (HFC-245fa) and uses thereof, including use in refrigerant compositions, refrigeration systems, blowing agent compositions, and aerosol propellants.

### FIELD OF THE INVENTION

The present invention provides azeotrope-like compositions of cis-1,3,3,3-tetrafluoropropene (HFO-1234ze(Z)) and 1,1,1,3,3-pentafluoropropane (HFC-245fa); and uses thereof, and in process for separating the azeotrope-like mixtures.

### BACKGROUND OF THE INVENTION

Fluorocarbon based fluids have found widespread use in industry in a number of applications, including as refrigerants, aerosol propellants, blowing agents, heat transfer media, power cycle fluids and gaseous dielectrics. Because of the suspected environmental problems associated with the use of some of these fluids, including the relatively high global warming potentials associated therewith, it is desirable to use fluids having low or even zero ozone depletion potential. Thus, the use of fluids that do not contain chlorofluorocarbons (“CFCs”) or hydrochlorofluorocarbons (“HCFCs”) is desirable.

Compounds having low ozone depletion potential include hydrofluorocarbons (“HFCs”), especially hydrofluoroolefins (“HFOs”). Compounds having a low global warming potential are also desirable. In this regard, the use of alkenes is also desirable due to their short atmospheric lifetime which results in a relatively low global warming potential. Additionally, the use of single component fluids or azeotropic mixtures, which do not fractionate on boiling and evaporation, is desirable. However, the identification of new, environmentally safe, non-fractionating mixtures is complicated due to the fact that azeotrope formation is not readily predictable.

The industry is continually seeking new fluorocarbon based mixtures that offer alternatives, and are considered environmentally safer substitutes for CFCs and HCFCs. Of particular interest are mixtures containing hydrofluorocarbons, fluoroolefins and other fluorinated compounds, which have a low ozone depletion potentials and low global warming potential. Such mixtures are the subject of this invention.

U.S. Patent Pub. No. 20080051611 shows a process for producing in which both trans-1,3,3,3-tetrafluoropropene (HFO-1234ze(E)) and HFO-1234ze(Z) are produced by dehydrofluorinating HFC-245fa. The first step of the process involves the catalytic conversion of HFC-245fa by dehydrofluorinating HFC-245fa to produce a product stream comprising a combination of HFO-1234ze(E), HFO-1234ze(Z), hydrogen fluoride (HF), and unreacted HFC-245fa. No azeotrope-like compositions of HFO-1234ze(Z) and HFC-245fa are taught or suggested therein.

The unpredictability regarding the formation of azeotrope and/or azeotrope-like compositions is well known. See for example, U.S. Pat. No. 3,085,065 (“It is impossible to predict that an azeotrope will form between any two compounds”); U.S. Pat. No. 5,182,040 (“it is not possible to predict the formation of azeotropes”); and U.S. Pat. No. 5,648,017 (“it is not possible to predict the formation of azeotropes”).

### SUMMARY OF THE INVENTION

The present invention is based upon the unexpected discovery that HFO-1234ze (Z) and HFC-245fa form azeotrope-like compositions.

This invention provides azeotrope-like compositions that help to satisfy the continuing need for alternatives to CFCs and HCFCs. The compositions of this invention exhibit relatively low global warming potentials (“GWP”). Accordingly, it has been recognized that such compositions can be used to great advantage in a number of applications, including as replacements for CFCs, HCFCs, and HFCs such as HFC-134a in refrigerant, aerosol, blowing agents, and other applications. In a preferred embodiment, this is accomplished by azeotrope-like compositions comprising, consisting essentially of, and/or consisting of effective amounts of 1234ze(Z) and HFC-245fa.

One embodiment of the invention is a blowing agent comprising, or consisting essentially of, one or more of the azeotrope-like composition of the present invention.

Another embodiment of the invention is a method of forming a foam comprising adding to a foamable composition a blowing agent comprising, or consisting essentially of one or more of the azeotrope-like compositions of the present invention. The blowing agent may further comprise a premix of a polyol and the blowing agent, wherein the blowing agent comprises an azeotrope-like composition of the present invention.

Yet another embodiment of the invention is a closed cell foam prepared by foaming a foamable composition in the presence of a blowing agent comprising, or consisting essentially of, one or more of the azeotrope-like compositions of the present invention. Preferably, the closed cell foam is formed from a foamable composition that further comprises polyurethane, polyisocyanurate, polystyrene, polyethylene, and mixtures thereof.

One embodiment of the invention is a refrigerant composition comprising, or consisting essentially of, one or more of the azeotrope-like compositions of the present invention. Another embodiment of the invention is a refrigeration system comprising a refrigerant composition of the present invention.

Yet another embodiment of the invention is a method for cooling an article which comprises evaporating a refrigerant composition of the invention in the vicinity of the article to be cooled. For example, a centrifugal chiller working fluid can be formed from an azeotrope-like composition of the present invention.

Another embodiment of the invention is a method for heating an article which comprises condensing a refrigerant composition comprising, or consisting essentially of, one or more of the azeotrope-like compositions of the present invention in the vicinity of the article to be heated.

One embodiment of the invention is a sprayable composition comprising a material to be sprayed and a propellant comprising, or consisting essentially of, one or more of the azeotrope-like compositions of the present invention.

Yet another embodiment of the invention is a composition useful for reducing the flammability of a fluid comprising one or more of the azeotrope-like compositions of the present invention added to said fluid.

Another embodiment of the invention is a composition useful for suppressing a flame comprising one or more of the azeotrope-like compositions of the present invention. Optionally, this fluid may further include one or more fluoroketone compounds. One preferred fluoroketone compound is dodecafluoro-2-methylpentan-3-one. One source for this compound is the 3M Company under the brand name Novec 1230.

Yet another embodiment of the invention is composition useful for cleaning and/or sterilizing an article, comprising, or consisting essentially of, one or more of the azeotrope-like compositions of the present invention. Optionally, the composition may further include ethylene oxide.

Another embodiment of the invention is directed to a method for the separation of the azeotropic compositions to yield essentially pure HFC-245fa and HFO-1234ze(Z), for example, by pressure swing distillation.

### DETAILED DESCRIPTION OF THE INVENTION

The present invention provides an azeotrope-like composition comprising cis-1,3,3,3-tetrafluoropropene (HFO-1234ze (Z)) and 1,1,1,3,3-pentafluoropropane (HFC-245fa).

The azeotrope-like compositions of the present invention exhibit properties that make that make them advantageous for use as, or in, a refrigerant, an aerosol, power cycle and blowing agent compositions.

### Azeotrope-Like Compositions

As used herein, the term “azeotrope-like” is intended in its broad sense to include both compositions that are strictly azeotropic and compositions that behave like azeotropic mixtures. From fundamental principles, the thermodynamic state of a fluid is defined by pressure, temperature, liquid composition, and vapor composition. An azeotropic mixture is a system of two or more components in which the liquid composition and vapor composition are equal at the stated pressure and temperature. In practice, this means that the components of an azeotropic mixture are constant-boiling and cannot be separated during a phase change.

The azeotrope-like compositions of the invention may include additional components that do not form new azeotrope-like systems, or additional components that are not in the first distillation cut. The first distillation cut is the first cut taken after the distillation column displays steady state operation under total reflux conditions. One way to determine whether the addition of a component forms a new azeotrope-like system so as to be outside of this invention is to distill a sample of the composition with the component under conditions that would be expected to separate a non-azeotropic mixture into its separate components. If the mixture containing the additional component is non-azeotrope-like, the additional component will fractionate from the azeotrope-like components. If the mixture is azeotrope-like, some finite amount of a first distillation cut will be obtained that contains all of the mixture components that is constant boiling or behaves as a single substance.

It follows from this that another characteristic of azeotrope-like compositions is that there is a range of compositions containing the same components in varying proportions that are azeotrope-like or constant boiling. All such compositions are intended to be covered by the terms “azeotrope-like” and “constant boiling”. As an example, it is well known that at differing pressures, the composition of a given azeotrope will vary at least slightly, as does the boiling point of the composition.

Thus, an azeotrope of A and B represents a unique type of relationship, but with a variable composition depending on temperature and/or pressure. It follows that, for azeotrope-like compositions, there is a range of compositions containing the same components in varying proportions that are azeotrope-like. All such compositions are intended to be covered by the term azeotrope-like as used herein. It is well-recognized in the art that it is not possible to predict the formation of azeotropes.

According to certain preferred embodiments, the azeotrope-like compositions of the present invention comprise, and preferably consist essentially of, and more preferably consist of, effective amounts of HFO-1234ze(Z) and HFC-245fa. The term “effective amounts” as used herein refers to the amount of each component which upon combination with the other component, results in the formation of an azeotrope-like composition of the present invention.

Any of a wide variety of methods known in the art for combining two or more components to form a composition can be adapted for use in the present methods to produce an azeotrope-like composition. For example, the first component and the second component can be mixed, blended, or otherwise contacted by hand and/or by machine, as part of a batch or continuous reaction and/or process, or via combinations of two or more such steps. In light of the disclosure herein, those of skill in the art will be readily able to prepare azeotrope-like compositions according to the present invention without undue experimentation.

In one aspect, an azeotrope-like composition is provided that consists essentially of, or consists of, HFO-1234ze(Z) and HFC-245fa. In one example, the HFC-245fa is in an amount from about 0.1 weight percent to about 25 weight percent based on the total weight of the azeotropic or azeotrope-like composition, the HFO-1234ze(Z) is in an amount from about 75 weight percent to about 99.9 weight percent based on the total weight of the azeotropic or azeotrope-like composition, and the azeotropic or azeotrope-like composition has a boiling point of about 9.1° C. at a pressure of about 14.4. More preferably the HFC-245fa is in an amount from about 1 weight percent to about 10 weight percent based on the total weight of the azeotropic or azeotrope-like composition, the HFO-1234ze(Z) is in an amount from about 90 weight percent to about 99 weight percent based on the total weight of the azeotropic or azeotrope-like composition. Most preferably the HFC-245fa is in an amount from about 5 weight percent to about 8 weight percent based on the total weight of the azeotropic or azeotrope-like composition, the HFO-1234ze(Z) is in an amount from about 95 weight percent to about 92 weight percent based on the total weight of the azeotropic or azeotrope-like composition.

The present compositions have utility in a wide range of applications. For example, one embodiment of the present invention relates to blowing agent, aerosol and cleaning, and refrigerant compositions comprising the present azeotrope-like compositions.

Yet another embodiment of the present invention relates to a blowing agent comprising one or more azeotrope-like compositions of the invention. One embodiment of the present invention relates to methods of forming thermoset foams, and preferably polyurethane and polyisocyanurate foams. The methods generally comprise providing a blowing agent composition of the present inventions, directly or indirectly adding the blowing agent composition to a foamable composition, and reacting the foamable composition under the conditions effective to form a foam or cellular structure, as is well known in the art. These comprise a foamable composition comprising the azeotrope-like composition above and at least one thermoset foam component. For example, the thermoset foam component may comprise a composition capable of forming polyurethane foam, a polyisocyanurate foam or a phenolic foam. It is possible to produce thermoplastic foams using the compositions of the invention. These foams may be open cell or closed cell. Any of the methods well known in the art, such as those described in “Polyurethanes Chemistry and Technology,” Volumes I and II, Saunders and Frisch, 1962, John Wiley and Sons, New York, N.Y., which is incorporated herein by reference, may be used or adapted for use in accordance with the foam embodiments of the present invention.

In general, polyurethane or polyisocyanurate foams are prepared by combining an isocyanate, the polyol premix composition, and other materials such as optional flame retardants, colorants, or other additives. These foams can be rigid, flexible, or semi-rigid, and can have a closed cell structure, an open cell structure or a mixture of open and closed cells. In general, such preferred methods comprise preparing polyurethane or polyisocyanurate foams by combining an isocyanate, a polyol or mixture of polyols, a blowing agent or mixture of blowing agents comprising one or more of the present compositions, and other materials such as catalysts, surfactants, and optionally, flame retardants, colorants, or other additives.

It is convenient in many applications to provide the components for polyurethane or polyisocyanurate foams in pre-blended formulations. Most typically, the foam formulation is pre-blended into two components. The isocyanate and optionally certain surfactants and blowing agents comprise the first component, commonly referred to as the “A” component. The polyol or polyol mixture, a surfactant including silicone surfactants, catalysts including amine catalysts, blowing agents, flame retardant, and other isocyanate reactive components comprise the second component, commonly referred to as the “B” component. The blowing agent comprises the azeotrope-like composition of this invention and optionally a hydrocarbon, fluorocarbon, chlorocarbon, fluorochlorocarbon, halogenated hydrocarbon, CO₂ generating material, or combinations thereof. The blowing agent component is usually present in the polyol premix composition in an amount of from about 1 wt. % to about 30 wt. %, by weight of the polyol premix composition. The polyol component, can be any polyol which reacts in a known fashion with an isocyanate in preparing polyurethane or polyisocyanurate foam. Useful polyols comprise one or more of a sucrose containing polyol; phenol, a phenol formaldehyde containing polyol; a glucose containing polyol; a sorbitol containing polyol; a methylglucoside containing polyol; an aromatic polyester polyol; glycerol; ethylene glycol; diethylene glycol; propylene glycol; graft copolymers of polyether polyols with a vinyl polymer; a copolymer of a polyether polyol with a polyurea; or combinations thereof.

The polyol component is usually present in the polyol premix composition in an amount of from about 60 wt. % to about 95 wt. %, by weight of the polyol premix composition. The polyol premix composition next contains a surfactant component which silicone surfactant and optionally an additional non-silicone surfactant. The surfactant is usually present in the polyol premix composition in an amount of from about 0.5 wt. % to about 5.0 wt. % by weight of the polyol premix composition. The polyol premix composition next contains a catalyst which is preferably an amine Tertiary amines are preferred. Preferred amines include: N,N-dimethylcyclohexyl-amine, dimethlyethanolamine, N,N,N′, N′,N″,N″-pentamethyldiethylenetriamine, 1,4-diaza-bicyclo[2.2.2]octane (DABCO), and triethylamine. The catalyst is usually present in the polyol premix composition in an amount of from about 0.1 wt. % to about 3.5 wt. % by weight of the polyol premix composition.

A foamable composition suitable for forming polyurethane or polyisocyanurate foam may be formed by reacting an organic polyisocyanate and the polyol premix composition described above. Any organic polyisocyanate can be employed in polyurethane or polyisocyanurate foam synthesis inclusive of aliphatic and aromatic polyisocyanates. Suitable organic polyisocyanates include aliphatic, cycloaliphatic, araliphatic, aromatic, and heterocyclic isocyanates which are well known in the field of polyurethane chemistry. These are described in, for example, U.S. Pat. Nos. 4,868,224; 3,401,190; 3,454,606; 3,277,138; 3,492,330; 3,001,973; 3,394,164; 3,124,605; and 3,201,372. Preferred as a class are the aromatic polyisocyanates. Representative organic polyisocyanates correspond to the formula:

R(NCO)z

wherein R is a polyvalent organic radical which is aliphatic, aralkyl, aromatic or mixtures thereof, and z is an integer which corresponds to the valence of R and is at least two.

Accordingly, polyurethane or polyisocyanurate foams are readily prepared by bringing together the A and B side components either by hand mix for small preparations and, preferably, machine mix techniques to form blocks, slabs, laminates, pour-in-place panels and other items, spray applied foams, froths, and the like. Optionally, other ingredients such as fire retardants, colorants, auxiliary blowing agents, and even other polyols can be added as a third stream to the mix head or reaction site. Most preferably, however, they are all incorporated into one B-component as described above. Conventional flame retardants can also be incorporated, preferably in amount of not more than about 20 percent by weight of the reactants.

In addition to the previously described ingredients, other ingredients such as, dyes, fillers, pigments and the like can be included in the preparation of the foams. Dispersing agents and cell stabilizers can be incorporated into the present blends. Conventional fillers for use herein include, for example, aluminum silicate, calcium silicate, magnesium silicate, calcium carbonate, barium sulfate, calcium sulfate, glass fibers, carbon black and silica. The filler, if used, is normally present in an amount by weight ranging from about 5 parts to 100 parts per 100 parts of polyol. A pigment which can be used herein can be any conventional pigment such as titanium dioxide, zinc oxide, iron oxide, antimony oxide, chrome green, chrome yellow, iron blue siennas, molybdate oranges and organic pigments such as para reds, benzidine yellow, toluidine red, toners and phthalocyanines.

The polyurethane or polyisocyanurate foams produced with the azeotrope-like composition of the present invention can vary in density from about 0.5 pounds per cubic foot to about 60 pounds per cubic foot, preferably from about 1.0 to 20.0 pounds per cubic foot, and most preferably from about 1.5 to 6.0 pounds per cubic foot. The density obtained is a function of how much of the blowing agent or blowing agent mixture disclosed in this invention plus the amount of auxiliary blowing agent, such as water or other co-blowing agents is present in the A and/or B components, or alternatively added at the time the foam is prepared. These foams can be rigid, flexible, or semi-rigid foams, and can have a closed cell structure, an open cell structure or a mixture of open and closed cells. These foams are used in a variety of well-known applications, including but not limited to thermal insulation, cushioning, flotation, packaging, adhesives, void filling, crafts and decorative, and shock absorption.

The invention also contemplates forming a thermoplastic form. For example, conventional polystyrene and polyethylene formulations may be combined with the azeotrope-like composition in a conventional manner to produce thermoplastic foams. Examples of thermoplastic foam components include polyolefins, such as for example polystyrene. Other examples of thermoplastic resins include polyethylene, ethylene copolymers, polypropylene, and polyethyleneterephthalate. In certain embodiments, the thermoplastic foamable composition is an extrudable composition. It is also generally recognized that the thermoplastic foamable composition may include adjuvants such as nucleating agents, flame or fire retardant materials, cell modifiers, cell pressure modifiers, and the like.

With respect to thermoplastic foams, the preferred methods generally comprise introducing a blowing agent in accordance with the present invention into a thermoplastic material, and then subjecting the thermoplastic material to conditions effective to cause foaming. For example, the step of introducing the blowing agent into the thermoplastic material may comprise introducing the blowing agent into a screw extruder containing a thermoplastic polymer, and the step of causing foam may comprise lowering the pressure on the thermoplastic material and thereby causing expansion of the blowing agent and contributing to the foaming of the material. Suitable thermoplastic polymers non-exclusively include polystyrene, polyethylene, polypropylene, polyethylene terephthalate, and combinations of these.

It will be generally appreciated by those skilled in the art, especially in view of the disclosure herein, that the order and manner in which the blowing agent of the present invention is formed and/or added to the foamable composition does not generally affect the operability of the present invention thermoset or thermoplastic foams. It is contemplated also that in certain embodiments it may be desirable to utilize the present compositions when in the supercritical or near supercritical state as a blowing agent.

The azeotrope-like compositions of this invention may also be used as refrigerant compositions. The refrigerant compositions of the present invention may be used in any of a wide variety of refrigeration systems including air-conditioning, refrigeration, heat-pump systems, and the like. In certain preferred embodiments, the compositions of the present invention are used in refrigeration systems originally designed for use with an HFC-refrigerant, such as, for example, HFC-134a. The preferred compositions of the present invention tend to exhibit many of the desirable characteristics of HFC-134a and other HFC-refrigerants, including non-flammability, and a GWP that is as low as, or lower than, that of conventional HFC-refrigerants. In addition, the relatively constant boiling nature of the compositions of the present invention makes them even more desirable than certain conventional HFCs for use as refrigerants in many applications.

In certain other preferred embodiments, the present compositions are used in refrigeration systems originally designed for use with a CFC-refrigerant. Preferred refrigeration compositions of the present invention may be used in refrigeration systems containing a lubricant used conventionally with CFC-refrigerants, such as mineral oils, silicone oils, and the like, or may be used with other lubricants traditionally used with HFC refrigerants. In certain embodiments, the compositions of the present invention may be used to retrofit refrigeration systems containing HFC, HCFC, and/or CFC-refrigerants and lubricants used conventionally therewith.

Preferably, the present methods involve recharging a refrigerant system that contains a refrigerant to be replaced and a lubricant comprising the steps of (a) removing the refrigerant to be replaced from the refrigeration system while retaining a substantial portion of the lubricant in said system; and (b) introducing to the system a composition of the present invention. As used herein, the term “substantial portion” refers generally to a quantity of lubricant which is at least about 50% by weight of the quantity of lubricant contained in the refrigeration system prior to removal of the chlorine-containing refrigerant. Preferably, the substantial portion of lubricant in the system according to the present invention is a quantity of at least about 60% of the lubricant contained originally in the refrigeration system, and more preferably a quantity of at least about 70%.

As used herein the term “refrigeration system” refers generally to any system or apparatus, or any part or portion of such a system or apparatus, which employs a refrigerant to provide cooling. Such refrigeration systems include, for example, air conditioners, electric refrigerators, chillers, transport refrigeration systems, commercial refrigeration systems and the like.

Any of a wide range of known methods can be used to remove refrigerants to be replaced from a refrigeration system while removing less than a major portion of the lubricant contained in the system. For example, because refrigerants are quite volatile relative to traditional hydrocarbon-based lubricants where the boiling points of refrigerants are generally less than 10° C. whereas the boiling points of mineral oils are generally more than 200° C. In embodiments wherein the lubricant is a hydrocarbon-based lubricant, the removal step may readily be performed by pumping chlorine-containing refrigerants in the gaseous state out of a refrigeration system containing liquid state lubricants. Such removal can be achieved in any of a number of ways known in the art, including, the use of a refrigerant recovery system, such as the recovery system manufactured by Robinair of Ohio.

Alternatively, a cooled, evacuated refrigerant container can be attached to the low pressure side of a refrigeration system such that the gaseous refrigerant is drawn into the evacuated container and removed. Moreover, a compressor may be attached to a refrigeration system to pump the refrigerant from the system to an evacuated container. In light of the above disclosure, those of ordinary skill in the art will be readily able to remove chlorine-containing lubricants from refrigeration systems and to provide a refrigeration system having therein a hydrocarbon-based lubricant and substantially no chlorine-containing refrigerant according to the present invention.

Any of a wide range of methods for introducing the present refrigerant compositions to a refrigeration system can be used in the present invention. For example, one method comprises attaching a refrigerant container to the low-pressure side of a refrigeration system and turning on the refrigeration system compressor to pull the refrigerant into the system. In such embodiments, the refrigerant container may be placed on a scale such that the amount of refrigerant composition entering the system can be monitored. When a desired amount of refrigerant composition has been introduced into the system, charging is stopped. Alternatively, a wide range of charging tools, known to those of skill in the art, is commercially available. Accordingly, in light of the above disclosure, those of skill in the art will be readily able to introduce the refrigerant compositions of the present invention into refrigeration systems according to the present invention without undue experimentation.

According to certain other embodiments, the present invention provides refrigeration systems comprising a refrigerant of the present invention and methods of producing heating or cooling by condensing and/or evaporating a composition of the present invention. In certain preferred embodiments, the methods for cooling an article according to the present invention comprise condensing a refrigerant composition comprising an azeotrope-like composition of the present invention and thereafter evaporating said refrigerant composition in the vicinity of the article to be cooled. Certain preferred methods for heating an article comprise condensing a refrigerant composition comprising an azeotrope-like composition of the present invention in the vicinity of the article to be heated and thereafter evaporating said refrigerant composition. In light of the disclosure herein, those of skill in the art will be readily able to heat and cool articles according to the present inventions without undue experimentation.

In another embodiment, the azeotrope-like compositions of this invention may be used as propellants in sprayable compositions, either alone or in combination with known propellants. The propellant composition comprises, more preferably consists essentially of, and, even more preferably, consists of the azeotrope-like compositions of the invention. The active ingredient to be sprayed together with inert ingredients, solvents, and other materials may also be present in the sprayable mixture. Preferably, the sprayable composition is an aerosol. Suitable active materials to be sprayed include, without limitation, cosmetic materials such as deodorants, perfumes, hair sprays, cleansers, defluxing agents, and polishing agents as well as medicinal materials such as anti-asthma and anti-halitosis medications. Other uses of the present azeotrope-like compositions include use as solvents, cleaning agents, and the like. Those skilled in the art will be readily able to adapt the present compositions for use in such applications without undue experimentation.

### EXAMPLES

The following non-limiting examples serve to further illustrate the invention.

### Example 1

An ebulliometer comprising a vacuum jacketed tube with a condenser on top which is further equipped with a Quartz Thermometer is used. About 17.36 g 1234ze (Z) is charged to the ebulliometer and then HFC-245fa is added in small, measured increments. Temperature depression is observed when HFC-245fa is added to 1234ze (Z), indicating a binary minimum boiling azeotrope is formed. From greater than about 0 to about 7 weight percent HFC-245fa, the boiling point of the composition stays below or around the boiling point of HFO-1234ze(Z).

The boiling temperature of 1234ze (Z) (99% pure) is about 9.12° C. at 14.4 psia, and the boiling of TOX grade HFO-1234ze(Z) (99.99% pure) is about 9.1° C. at 14.4 psia. The boiling point of HFC-245fa is about 15° C. at 14.4 psia. The binary mixtures shown in Table 1 were studied. The temperature increases by <1° C. when HFC-245fa is added up to a concentration of 25.6 wt %. The compositions exhibit azeotrope and/or azeotrope-like properties over this range.

TABLE 1
HFO-1234ze (Z)/HFC-245fa Compositions at P = 14.4 psia.

| T(° C.) | Wt. % HFO-1234ze(Z) | Wt. % HFC-245fa |
|---|---|---|
| 9.1 | 100 | 0.0 |
| 9.1 | 99.60 | 0.4 |
| 9.2 | 98.52 | 1.48 |
| 9.3 | 93.23 | 6.77 |
| 9.3 | 88.66 | 11.34 |
| 9.6 | 83.70 | 16.30 |
| 9.7 | 78.69 | 21.31 |
| 9.9 | 74.38 | 25.62 |

### Example 2

A mixture of 93% HFO-1234ze(Z) and 7% HFC-245fa was placed in the reboiler of a distillation column. A distillation column was constructed of Monel. The column consisted of a 1 L reboiler attached to a 1 in internal diameter by 4 foot tall column. The column was packed with heli-pak high efficiency packing which was rated at 80 theoretical staged for this design. The column temperature and pressure was controlled by the flow rate of liquid nitrogen to the condenser. The distillate was taken from the vapor overhead of the distillation column. Prior to use the distillation column was flushed with clean dry nitrogen and then evacuated.

The column was operated at 7.0° C. overhead temperature, 14.4 psia and a differential pressure between the top and bottom of the column of 0.6 in of water. There were 7 main cuts taken and the summary of the cuts are given in Table 2. This Table shows that the composition of the overhead stayed nearly constant at 95% HFO-1234ze(Z) indicating that an azeotrope-like composition was formed. The azeotrope-like composition was shown to be at ˜7% HFO-1234ze(Z) by this distillation.

TABLE 2
Distillation of HFO-1234ze(Z) and HFC-245fa

Concentration

| | HFO- | HFC- | Amount Collected |
|---|---|---|---|
| | 1234ze(Z) | 245fa | grams |
| Cut #1 | 73% | 27% | 83 |
| Cut #2 | 91% | 9% | 100 |
| Cut #3 | 90% | 10% | 123 |
| Cut #4 | 91% | 9% | 115 |
| Cut #5 | 93% | 7% | 140 |
| Cut #6 | 93% | 7% | 140 |
| Cut #7 | 93% | 7% | 147 |

### Examples of Uses of the Compositions

As described above, the azeotrope-like compositions of the present invention may be used in a wide variety of applications as substitutes for CFCs and for compositions containing less desirable HCFCs.

Specifically, the azeotrope-like compositions of the present invention may be used as blowing agents, refrigerants, heating agents, power cycle agents, cleaning agents, aerosol propellants, sterilization agents, lubricants, flavor and fragrance extractants, flammability reducing agents, and flame suppression agents, to name a few preferred uses.

The azeotrope-like compositions of the invention are also useful in connection with numerous methods and systems, including as heat transfer fluids in methods and systems for transferring heat, such as refrigerants used in refrigeration, air conditioning and heat pump systems.

The present azeotrope-like compositions are also advantageous for in use in systems and methods of generating aerosols, preferably comprising or consisting of the aerosol propellant in such systems and methods. Methods of methods of extinguishing and suppressing fire are also included in certain aspects of the present invention. The present invention also provides in certain aspects methods of removing residue from articles in which the present azeotrope-like compositions are used as solvent compositions in such methods and systems.

### Example 3

### Blowing Agents

One embodiment of the present invention relates to a blowing agent comprising one or more of the azeotrope-like compositions of the invention. In other embodiments, the invention provides foamable compositions, and preferably polyurethane and polyisocyanurate foam compositions, and methods of preparing foams. In such foam embodiments, one or more of the azeotrope-like compositions, are included as a blowing agent in a foamable composition, which composition preferably includes one or more additional components capable of reacting and foaming under the proper conditions to form a foam or cellular structure, as is well known in the art.

The present methods preferably comprise providing such a foamable composition and reacting it under conditions effective to obtain a foam, and preferably a closed cell foam. The invention also relates to foam, and preferably closed cell foam, prepared from a polymer foam formulation containing a blowing agent comprising an azeotrope-like composition of the invention.

In certain embodiments, one or more of the following HFC isomers are preferred for use as co-blowing agents in the azeotrope-like compositions of the present invention:
1,1,1,2,2-pentafluoroethane (HFC-125)
1,1,2,2-tetrafluoroethane (HFC-134)
1,1,1,2-tetrafluoroethane (HFC-134a)
1,1-difluoroethane (HFC-152a)
1,1,1,2,3,3,3-heptafluoropropane (HFC-227ea)
1,1,1,3,3,3-hexafluoropropane (HFC-236fa)
1,1,1,3,3-pentafluoropropane (HFC-245fa) and
1,1,1,3,3-pentafluorobutane (HFC-365mfc).

The relative amount of any of the above noted additional co-blowing agents, as well as any additional components which may be included in the present azeotrope-like compositions, can vary widely within the general broad scope of the present invention according to the particular application for the composition, and all such relative amounts are considered to be within the scope hereof.

### Example 4

### Foams

The present invention also relates to all foams, including but not limited to closed cell foam, open cell foam, rigid foam, flexible foam, integral skin and the like, prepared from a polymer foam formulation containing a blowing agent comprising an azeotrope-like composition of the present invention. Although it is contemplated that the present foams, particularly thermoset foams of the present invention, may be used in a wide variety of applications, in certain preferred embodiments the present invention comprises appliance foams in accordance with the present invention, including refrigerator foams, freezer foams, refrigerator/freezer foams, panel foams, and other cold or cryogenic manufacturing applications.

The foams prepared in accordance with the present invention, in certain preferred embodiments, provide one or more exceptional features, characteristics and/or properties, including: thermal insulation efficiency (particularly for thermoset foams), dimensional stability, compressive strength, aging of thermal insulation properties, all in addition to the low ozone depletion potential and low global warming potential associated with many of the preferred blowing agents of the present invention.

### Example 5

### Heat Transfer Methods

The preferred heat transfer methods generally comprise providing an azeotrope-like composition of the present invention and causing heat to be transferred to or from the composition by changing the phase of the composition. For example, the present methods provide cooling by absorbing heat from a fluid or article, preferably by evaporating the present refrigerant composition in the vicinity of the body or fluid to be cooled to produce vapor comprising the present composition.

Preferably the methods include the further step of compressing the refrigerant vapor, usually with a compressor or similar equipment to produce vapor of the present composition at a relatively elevated pressure. Generally, the step of compressing the vapor results in the addition of heat to the vapor, thus causing an increase in the temperature of the relatively high-pressure vapor.

Preferably, the present methods include removing from this relatively high temperature, high pressure vapor at least a portion of the heat added by the evaporation and compression steps. The heat removal step preferably includes condensing the high temperature, high-pressure vapor while the vapor is in a relatively high-pressure condition to produce a relatively high-pressure liquid comprising a composition of the present invention. This relatively high-pressure liquid preferably then undergoes a nominally isoenthalpic reduction in pressure to produce a relatively low temperature, low-pressure liquid. In such embodiments, it is this reduced temperature refrigerant liquid which is then vaporized by heat transferred from the body or fluid to be cooled.

In another process embodiment of the invention, the azeotrope-like compositions of the invention may be used in a method for producing heating which comprises condensing a refrigerant composition comprising, or consisting essentially of, the azeotrope-like compositions of this invention, in the vicinity of a liquid or body to be heated. Such methods, as mentioned hereinbefore, frequently are reverse cycles to the refrigeration cycle described above.

### Example 6

### Refrigerant Compositions

The azeotrope-like compositions of the present invention are adaptable for use in connection with automotive air conditioning systems and devices, commercial refrigeration systems and devices, chillers, residential refrigerator and freezers, general air conditioning systems, heat pumps, and the like.

Many existing refrigeration systems are currently adapted for use in connection with existing refrigerants, and the azeotrope-like compositions of the present invention are believed to be adaptable for use in many of such systems, either with or without system modification. In many applications the azeotrope-like compositions of the present invention may provide an advantage as a replacement in systems, which are currently based on refrigerants having a relatively high capacity. Furthermore, in embodiments where it is desired to use a lower capacity refrigerant composition of the present invention, for reasons of efficiency for example, to replace a refrigerant of higher capacity, such embodiments of the present compositions provide a potential advantage.

### Example 7

### Lubricant Compositions

In certain embodiments, the azeotrope-like compositions of the present invention can further comprise a lubricant. Any of a variety of conventional lubricants may be used in the azeotrope-like compositions of the present invention. An important requirement for the lubricant is that, when in use in a refrigerant system, there must be sufficient lubricant returning to the compressor of the system such that the compressor is lubricated. Thus, suitability of a lubricant for any given system is determined partly by the refrigerant/lubricant characteristics and partly by the characteristics of the system in which it is intended to be used.

Examples of suitable lubricants include mineral oil, alkyl benzenes, polyol esters, including polyalkylene glycols, PAG oil, and the like. Mineral oil, which comprises paraffin oil or naphthenic oil, is commercially available. Commercially available mineral oils include Witco LP 250 (registered trademark) from Witco, Zerol 300 (registered trademark) from Shrieve Chemical, Sunisco 3GS from Witco, and Calumet R015 from Calumet. Commercially available alkyl benzene lubricants include Zerol 150 (registered trademark). Commercially available esters include neopentyl glycol dipelargonate which is available as Emery 2917 (registered trademark) and Hatcol 2370 (registered trademark). Other useful esters include phosphate esters, dibasic acid esters, and fluoroesters. Preferred lubricants include polyalkylene glycols and esters. Certain more preferred lubricants include polyalkylene glycols.

### Example 8

### Power Cycle Fluids

Organic Rankine cycle systems are often used to recover waste heat from industrial processes. In combined heat and power (cogeneration) applications, waste heat from combustion of fuel used to drive the prime mover of a generator set is recovered and used to make hot water for building heat, for example, or for supplying heat to operate an absorption chiller to provide cooling. In some cases, the demand for hot water is small or does not exist. The most difficult case is when the thermal requirement is variable and load matching becomes difficult, confounding efficient operation of the combined heat and power system. In such an instance, it is more useful to convert the waste heat to shaft power by using an organic Rankine cycle system. The shaft power can be used to operate pumps, for example, or it may be used to generate electricity. By using this approach, the overall system efficiency is higher and fuel utilization is greater. Air emissions from fuel combustion can be decreased since more electric power can be generated for the same amount of fuel input.

The process that produces waste heat is at least one selected from the group consisting of fuel cells, internal combustion engines, internal compression engines, external combustion engines, and turbines. Other sources of waste heat can be found in association with operations at oil refineries, petrochemical plants, oil and gas pipelines, chemical industry, commercial buildings, hotels, shopping malls, supermarkets, bakeries, food processing industries, restaurants, paint curing ovens, furniture making, plastics molders, cement kilns, lumber kilns (drying), calcining operations, steel industry, glass industry, foundries, smelting, air-conditioning, refrigeration, and central heating. See U.S. Pat. No. 7,428,816, the disclosure of which is hereby incorporated herein by reference.

Rankine cycle systems are known to be a simple and reliable means to convert heat energy into mechanical shaft power. Organic working fluids are useful in place of water/steam when low-grade thermal energy is encountered. Water/steam systems operating with low-grade thermal energy (typically 400° F. and lower) will have associated high volumes and low pressures. To keep system size small and efficiency high, organic working fluids with boiling points near room temperature are employed.

The azeotrope-like compositions of the present invention are suitable for use as organic working fluids, providing higher gas densities lending to higher capacity and favorable transport and heat transfer properties lending to higher efficiency as compared to water at low operating temperatures.

### Example 9

### Cleaning Fluids

The azeotrope-like compositions of the present invention are also useful for removing containments from a product, part, component, substrate, or any other article or portion thereof by applying to the article an azeotrope-like composition of the present invention. For the purposes of convenience, the term “article” is used herein to refer to all such products, parts, components, substrates, and the like and is further intended to refer to any surface or portion thereof. Furthermore, the term “contaminant” is intended to refer to any unwanted material or substance present on the article, even if such substance is placed on the article intentionally. For example, in the manufacture of semiconductor devices it is common to deposit a photoresist material onto a substrate to form a mask for the etching operation and to subsequently remove the photoresist material from the substrate. The term “contaminant” as used herein is intended to cover and encompass such a photo resist material.

Preferred cleaning methods of the present invention comprise applying the present composition to the article. Although it is contemplated that numerous and varied cleaning techniques can employ the azeotrope-like compositions of the present invention to good advantage, it is considered to be particularly advantageous to use the present compositions in connection with supercritical cleaning techniques. Supercritical cleaning is disclosed in U.S. Pat. No. 6,589,355, which is hereby incorporated herein by reference.

For supercritical cleaning applications, it is preferred in certain embodiments to include in the present cleaning compositions, in addition to the azeotrope-like composition of the present invention, another component, such as CO2 and other additional components known for use in connection with supercritical cleaning applications.

It may also be possible and desirable in certain embodiments to use the present cleaning compositions in connection with particular sub-critical vapor degreasing and solvent cleaning methods.

Another cleaning embodiment of the invention comprises the removal of contaminants from vapor compression systems and their ancillary components when these systems are manufactured and serviced. As used herein, the term “contaminants” refers to processing fluids, lubricants, particulates, sludge, and/or other materials that are used in the manufacture of these systems or generated during their use. In general, these contaminants comprise compounds such as alkylbenzenes, mineral oils, esters, polyalkyleneglycols, polyvinylethers and other compounds that are made primarily of carbon, hydrogen and oxygen. The azeotrope-like compositions of the present invention will be useful for this purpose.

### Example 10

### Sterilization

The azeotrope-like compositions of this invention may be used as sterilization compositions, either alone or in combination with known sterilization materials. Many articles, devices and materials, particularly for use in the medical field, must be sterilized prior to use for the health and safety reasons, such as the health and safety of patients and hospital staff. The present invention provides methods of sterilizing comprising contacting the articles, devices or material to be sterilized with an azeotrope-like composition of the present invention, and optionally in combination with one or more additional sterilizing agents.

While many sterilizing agents are known in the art and are considered to be adaptable for use in connection with the present invention, in certain preferred embodiments sterilizing agent comprises ethylene oxide, formaldehyde, hydrogen peroxide, chlorine dioxide, ozone and combinations of these. In certain embodiments, ethylene oxide is the preferred sterilizing agent. Those skilled in the art, in view of the teachings contained herein, will be able to readily determine the relative proportions of sterilizing agent and the azeotrope-like compositions of the present invention to be used in sterilizing compositions and methods, and all such ranges are within the broad scope hereof.

As is known to those skilled in the art, certain sterilizing agents, such as ethylene oxide, are extremely flammable components, and the compound(s) in accordance with the present invention are included in the present compositions in amounts effective, together with other components present in the composition, to reduce the flammability of the sterilizing composition to acceptable levels. The sterilization methods of the present invention may be either high or low-temperature sterilization of the present invention involves the use of a compound or composition of the present invention at a temperature of from about 250° F. to about 270° F., preferably in a substantially sealed chamber. The process can be completed usually in less than about two hours. However, some articles, such as plastic articles and electrical components, cannot withstand such high temperatures and require low-temperature sterilization.

### Example 11

### Propellants

The azeotrope-like compositions of this invention may be used as propellants in sprayable compositions, either alone or in combination with known propellants. The propellant composition comprises, more preferably consists essentially of, and, even more preferably, consists of the azeotrope-like compositions of the invention. The active ingredient to be sprayed together with inert ingredients, solvents, and other materials may also be present in the sprayable mixture. Preferably, the sprayable composition is an aerosol. Suitable active materials to be sprayed include, without limitation, lubricants, insecticides, cleaners, cosmetic materials such as deodorants, perfumes and hair sprays, polishing agents, as well as medicinal materials such as skin cooling agents (sunburn treatment), topical anesthetics and anti-asthma medications.

The sprayable composition includes a material to be sprayed and a propellant including the azeotrope-like compositions of this invention. Inert ingredients, solvents, and other materials may also be present in the sprayable mixture. Preferably, the sprayable composition is an aerosol. Suitable materials to be sprayed include, without limitation, cosmetic materials such as deodorants, perfumes, hair sprays, cleansers, and polishing agents as well as medicinal materials such as anti-asthma and anti-halitosis medications.

### Example 12

### Extraction Compositions

The azeotrope-like compositions of the present invention also provide advantage when used to carry, extract or separate desirable materials from biomass. These materials include, but are not limited to, essential oils such as flavors and fragrances, oils which may be used as fuel, medicinals, nutraceuticals, etc.

### Example 13

### Flame Reduction Compositions

The azeotrope-like compositions of the present invention also provide advantage when used for reducing the flammability of fluids, said methods comprising adding an azeotrope-like composition of the present invention to said fluid. The flammability associated with any of a wide range of otherwise flammable fluids may be reduced according to the present invention. For example, the flammability associated with fluids such as ethylene oxide, flammable hydrofluoro-carbons and hydrocarbons, including: 1,1,1-trifluoroethane, difluoromethane, propane, hexane, octane, and the like, can be reduced according to the present invention. For the purposes of the present invention, a flammable fluid may be any fluid exhibiting flammability ranges in air as measured via any standard conventional test method, such as ASTM E-681, and the like.

Any suitable amounts of the present azeotrope-like compositions may be added to reduce flammability of a fluid according to the present invention. As will be recognized by those of skill in the art, the amount added will depend, at least in part, on the degree to which the subject fluid is flammable and the degree to which it is desired to reduce the flammability thereof. In certain preferred embodiments, the amount of the azeotrope-like composition added to the flammable fluid is effective to render the resulting fluid substantially non-flammable.

The azeotrope-like compositions of the present invention also provide advantage when used for suppressing a flame, wherein said methods comprising contacting a flame with an azeotrope-like composition of the present invention. If desired, additional flame suppressing agents can also be used with the azeotrope-like composition of the present invention, either in admixture, or as a secondary flame suppressing agent. One class of compounds useful for this purpose is the fluoroketones. One especially preferred fluoroketone is dodecafluoro-2-methyl-pentan-3-one. One commercial source for this preferred compound is the 3M Company under the trade name Novec 1230.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

While the present invention has been particularly shown and described with reference to preferred embodiments, it will be readily appreciated by those of ordinary skill in the art that various changes and modifications may be made without departing from the scope of the invention. It is intended that the claims be interpreted to cover the disclosed embodiment, those alternatives which have been discussed above and all equivalents thereto.

What is claimed is:

1. An azeotrope-like composition comprising effective amounts of HFO-1234ze(Z) and HFC-245fa.

2. The azeotrope-like composition of claim 1, wherein the HFC-245fa is present in an amount from about 0.1 weight percent to about 25 weight percent based on the total weight of the composition.

3. The azeotrope-like composition of claim 1, wherein the HFO-1234ze(Z) is in an amount from about 75 weight percent to about 99.9 weight percent based on the total weight of the composition.

4. The azeotrope-like composition of claim 1, wherein the HFC-245fa is in an amount from about 1 weight percent to about 10 weight percent based on the total weight of the composition.

5. The azeotrope-like composition of claim 1, wherein the HFO-1234ze(Z) is in an amount from about 90 weight percent to about 99 weight percent based on the total weight of the composition.

6. The azeotrope-like composition of claim 1, wherein the HFC-245fa is in an amount from about 5 weight percent to about 8 weight percent based on the total weight of the composition.

7. The azeotrope-like composition of claim 1, wherein the HFO-1234ze(Z) is in an amount from about 95 weight percent to about 92 weight percent based on the total weight of the composition.

8. The azeotrope-like composition of claim 1, wherein the azeotrope-like composition has a boiling point of about 9.1° C. at a pressure of about 14.4 psig.

9. An azeotrope-like composition consisting essentially of effective amounts of HFO-1234ze(Z) and HFC-245fa.

10. The azeotrope-like composition of claim 9, wherein the HFC-245fa is present in an amount from about 0.1 weight percent to about 25 weight percent based on the total weight of the composition.

11. The azeotrope-like composition of claim 9, wherein the HFO-1234ze(Z) is in an amount from about 75 weight percent to about 99.9 weight percent based on the total weight of the composition.

12. The azeotrope-like composition of claim 9, wherein the HFC-245fa is in an amount from about 1 weight percent to about 10 weight percent based on the total weight of the composition.

13. The azeotrope-like composition of claim 9, wherein the HFO-1234ze(Z) is in an amount from about 90 weight percent to about 99 weight percent based on the total weight of the composition.

14. The azeotrope-like composition of claim 9, wherein the HFC-245fa is in an amount from about 5 weight percent to about 8 weight percent based on the total weight of the composition.

15. The azeotrope-like composition of claim 9, wherein the HFO-1234ze(Z) is in an amount from about 95 weight percent to about 92 weight percent based on the total weight of the composition.

16. An azeotrope-like composition consisting of effective amounts of HFO-1234ze(Z) and HFC-245fa.

17. The azeotrope-like composition of claim 16, wherein the HFC-245fa is present in an amount from about 0.1 weight percent to about 25 weight percent based on the total weight of the composition.

18. The azeotrope-like composition of claim 16, wherein the HFO-1234ze(Z) is in an amount from about 75 weight percent to about 99.9 weight percent based on the total weight of the composition.

19. The azeotrope-like composition of claim 16, wherein the HFC-245fa is in an amount from about 1 weight percent to about 10 weight percent based on the total weight of the composition.

20. The azeotrope-like composition of claim 16, wherein the HFO-1234ze(Z) is in an amount from about 90 weight percent to about 99 weight percent based on the total weight of the composition.

21. The azeotrope-like composition of claim 16, wherein the HFC-245fa is in an amount from about 5 weight percent to about 8 weight percent based on the total weight of the composition.

22. The azeotrope-like composition of claim 16, wherein the HFO-1234ze(Z) is in an amount from about 95 weight percent to about 92 weight percent based on the total weight of the composition.

23. A blowing agent comprising an azeotrope-like composition of claim 1.

24. A method of forming a foam comprising adding to a foamable composition a blowing agent comprising an azeotrope-like composition of claim 1.

25. A premix of a polyol and a blowing agent wherein the blowing agent comprises an azeotrope-like composition of claim 1.

26. A closed cell foam prepared by foaming a foamable composition in the presence of a blowing agent comprising the azeotrope-like composition of claim 1.

27. The closed cell foam of claim 25, wherein said foamable composition comprises polyurethane, polyisocyanurate, polystyrene, polyethylene, and mixtures thereof.

28. A refrigerant composition comprising an azeotrope-like composition of claim 1.

29. A refrigeration system comprising a refrigerant composition of claim 28.

30. A method for cooling an article which comprises evaporating a refrigerant composition of claim 28 in the vicinity of the article to be cooled.

31. A method for heating an article which comprises condensing a refrigerant composition of claim 28 in the vicinity of the article to be heated.

32. A sprayable composition comprising a material to be sprayed and a propellant comprising an azeotrope-like composition of claim 1.

33. A method for sterilizing an article which comprises treating the article to be sterilized with an azeotrope-like composition of claim 1.

34. A lubricant composition comprising an azeotrope-like composition of claim 1.

35. A method of extracting flavors or fragrances from biomass which comprises treating the biomass with an azeotrope-like composition of claim 1.

36. A method of sterilizing an article, said method comprising contacting said article to be sterilized with a composition comprising an azeotrope-like composition of claim 1.

37. The method of claim 36, wherein said composition further comprises ethylene oxide.

38. A method of reducing the flammability of a fluid comprising adding an azeotrope-like composition of claim 1 to said fluid.

39. A method of suppressing a flame comprising contacting said flame with a fluid comprising an azeotrope-like composition of claim 1.

40. The method of claim 39, wherein the azeotrope-like composition further comprises a fluoroketone compound.

41. The method of claim 40, wherein the fluoroketone compound is dodecafluoro-2-methylpentan-3-one.

42. A centrifugal chiller working fluid comprising an azeotrope-like composition of claim 1.

43. A power cycle working fluid comprising an azeotrope-like composition of claim 1.
